(19) United States
(12) Patent Application Publication
(10) Pub. No.: US 2006/0004241 A1
(43) Pub. Date: Jan. 5, 2006

(54) Isothermal method for the dehydrogenating alkanes

(76) Inventors: Gotz Peter Schindler, Mannheim (DE); Klaus Harth, Altleiningen (DE)

Correspondence Address: CONNOLLY BOVE LODGE & HUTZ LLP, SUITE 800, 1990 M STREET NW, WASHINGTON, DC, 20036-3425, US

(21) Appl. No.: 10/524,133

(22) PCT Filed: Aug. 14, 2003
(86) PCT No.: PCT/EP03/09057

§ 371 Date: Feb. 11, 2005

(30) Foreign Application Priority Data
Aug. 16, 2002 (DE) 102-37-514.3

(51) Int. Cl. C07C 5/327

(52) U.S. Cl. 585/654

(57) ABSTRACT

Isothermal process for the dehydrogenation of alkanes to the corresponding alkenes over a catalyst bed comprising a dehydrogenation-active catalyst, wherein the catalyst bed comprises a catalytically inactive, inert diluent material. The catalytically inactive, inert diluent material is preferably selected from the group consisting of the oxides of the elements of main groups II, III and IV, transition groups III and IV and V, mixtures thereof and nitrides and carbides of elements of main groups III and IV and preferably has a BET surface area of <10 m²/g.

The presence of the catalytically inactive diluent material in the catalyst bed limits the space-time yield, based on alkene formed to preferably 7.0 kg/(kg_{bed}×h).

The present invention relates to an isothermal process for the dehydrogenation of alkanes to alkenes, in particular an isothermal process for the dehydrogenation of propane to propene.

The dehydrogenation of propane to propene is strongly exothermic with a reaction enthalpy ΔH of 135 kJ/mol. Propane and propene have only a comparatively low heat capacity of 160 J/(mol×K) or 135 J/(mol×K) at 600° C. In the dehydrogenation of propane, this leads to high temperature gradients within the dehydrogenation reactor, as a result of which the reaction is greatly limited by heat transport.

Adiabatic processes such as the UOP Oleflex avoid heat transport limitation of the dehydrogenation reaction, i.e. limitation by heat transport from the reactor walls into the interior of the reactor, by the required heat of reaction being made available in the form of the heat stored in the superheated incoming gas. Up to 4 reactors are typically connected in series. The incoming gas is superheated to 300 K upstream of its reactor. The use of a plurality of reactors enables excessively large differences in the temperatures of the reaction gas mixture between reactor inlet and reactor outlet to be avoided. The superheating of the incoming gas mixture results, firstly, in formation of carbon precursors which cause carbonization of the catalyst and, secondly, in a reduction in the selectivity of propane dehydrogenation due to cracking processes (formation of methane and ethene).

The high degree of superheating of the incoming gases is avoided in the isothermal processes of Linde and Krupp/Uhde (STAR process) by use of directly fired reactor tubes. Here, the feed gas mixture is heated only to the reaction temperature and the energy required for the endothermic reaction is introduced into the system over the entire length of the reactor via the reactor wall, with an isothermal temperature profile being sought both in the axial direction and in the radial direction. To avoid the formation of carbon precursors in the preheating of the incoming gas mixture, the incoming gas mixture can also be fed to the reactor at a lower temperature than the temperature required for the reaction, and not only the heat required for the endothermic reaction but also the additional heat required for heating the reaction mixture to the reaction temperature can be introduced into the reaction gas via the reactor wall.

However, in the isothermal propane dehydrogenation carried out in practice on an industrial scale, a temperature profile which deviates to a sometimes high degree from the ideal temperature profile is obtained. Particularly in the inlet region of the catalyst bed, i.e. where the system is still far from thermodynamic equilibrium and large incremental conversions are achieved, high temperature gradients occur both in an axial direction and in a radial direction. The lowest temperatures occur where the greatest conversions per unit volume are achieved.

It is an object of the present invention to provide an improved isothermal process for the dehydrogenation of propane to propene. In particular, it is an object of the invention to provide a process of this type in which the heat transport limitation in the catalyst bed is reduced and the occurrence of high temperature gradients in the catalyst bed is avoided.

We have found that this object is achieved by an isothermal process for the dehydrogenation of alkanes to the corresponding alkenes over a catalyst bed comprising a dehydrogenation-active catalyst, wherein the catalyst bed comprises an inert, catalytically inactive diluent material.

In the following, an isothermal process is, in contrast to an adiabatic process, a process in which heat is introduced from the outside into the reacting gas mixture by heating the reactor externally.

The catalyst bed is preferably diluted with catalytically inactive inert material at those places at which large axial and/or radial temperature gradients would be established without such dilution. This is particularly the case at places in the catalyst bed where high incremental conversions are achieved, i.e. particularly in the inlet region of the dehydrogenation reactor.

Suitable catalytically inactive inert materials are, for example, the oxides of elements of main groups II, III and IV, transition groups III, IV and V and also mixtures of two or more of these oxides, and also nitrides and carbides of elements of main groups III and IV. Examples are magnesium oxide, aluminum oxide, silicon dioxide, steatite, titanium dioxide, zirconium dioxide, niobium oxide, thorium oxide, aluminum nitride, silicon carbide, magnesium silicates, aluminum silicates, clay, kaolin and pumice. The catalytically inactive inert diluent materials preferably have a low BET surface area. This is generally <10 m²/g, preferably <5 m²/g and particularly preferably <1 m²/g. A low BET surface area can be obtained by ignition of the abovementioned oxides or ceramic materials at high temperatures of, for example, >1 000° C.

The catalytically inactive, inert diluent material preferably has a coefficient of thermal conduction at 293 K of >0.04 W/(m×K), preferably >0.4 W/(m×K) and particularly preferably >2 W/(m×K). The radial thermal conductivity of the catalyst bed diluted with catalytically inactive inert material is preferably >2 W/(m×K), particularly preferably >6 W/(m×K), in particular >10 W/(m×K).

The catalytically inactive, inert diluent material can be used in the form of crushed material or shaped bodies. The geometry and dimensions of the catalytically inactive diluent material are preferably chosen so that the diluent material and the dehydrogenation-active catalyst mix readily. This is generally the case when catalyst particles and the particles of catalytically inactive diluent material have approximately the same particle diameter.

The geometry of the particles of catalytically inactive diluent material can be selected so that the pressure drop established over the total length of the bed is less than the pressure drop which would be established over an undiluted bed containing the same amount of dehydrogenation-active catalyst. For example, rings or hollow extrudates of catalytically inactive diluent material can be used for this purpose. These also effect the improved temperature uniformity (isothermal nature) since they force the gas flowing through to flow in a direction which deviates from the main axial direction of the reactor tubes. The resulting improved convecting mixing increases the heat transport in the reaction gas mixture. As a result, the pressure drop is reduced and the radial thermal conductivity increases with increasing size of the rings or hollow extrudates. However, the use of excessively large shaped bodies is less preferred because of the poor mixing with the (smaller) catalyst particles which then results. Small catalyst particles are preferred over large catalyst particles because of the mass transport limitation which otherwise occurs.

Examples of suitable shaped body geometries are pellets or extrudates having an average diameter of from 2 to 8 mm and an average height of from 2 to 16 mm. The height is preferably from 0.5 to 4 times the diameter, particularly preferably 1 to 2 times the diameter.

Also suitable are rings or hollow extrudates having an average external diameter of from 6 to 20 mm and an average height of from 6 to 20 mm. The height is preferably from 0.5 to 4 times the diameter, particularly preferably about 1-2 times the diameter. The wall thickness is usually from 0.1 to 0.25 times the diameter. As indicated above, the rings and hollow extrudates have the additional advantage of better convective mixing of the reaction gas mixture and, in particular, a lower pressure drop. The pressure drop in the diluted bed can be even lower than that in an undiluted bed despite the increased volume and thus an increased reactor length.

A further suitable geometry of the shaped bodies is a spherical geometry. Spheres preferably have an average diameter of from 1 to 5 mm.

In particular, shaped catalyst bodies and shaped bodies of inert material have similar or even identical geometry and dimensions.

The proportion of empty space in the catalyst bed diluted with the catalytically inactive diluent material is preferably at least 30%, more preferably from 30 to 70%, particularly preferably from 40 to 70%.

The hydrogenation-active catalyst and catalytically inactive inert diluent material are generally present in a ratio of catalyst:inert material of from 0.01:1 to 10:1, preferably from 0.1:1 to 2:1, in each case based on the bed volumes of catalyst and inert material.

A suitable form of reactor for carrying out the alkane dehydrogenation of the present invention is a fixed-bed tube reactor or a shell-and-tube reactor. In the case of these reactors, the catalyst (dehydrogenation catalyst and, when using oxygen as cofeed, possibly a specific oxidation catalyst) is located as a fixed bed in a reaction tube or in a bundle of reaction tubes. The reaction tubes are usually indirectly heated by a gas, e.g. a hydrocarbon such as methane, being burnt in the space surrounding the reaction tubes. It is advantageous to employ this indirect form of heating only along the first about 20-30% of the length of the fixed bed and to heat the remaining length of the bed to the required reaction temperature by the radiative heat emitted by the indirect heating. Customary internal diameters of the reaction tubes are from about 10 to 15 cm. A typical shell-and-tube dehydrogenation reactor has from about 300 to 1 000 reaction tubes. The temperature in the interior of the reaction tubes usually ranges from 300 to 700° C., preferably from 400 to 700° C. The working pressure is usually in the range from 0.5 to 12 bar, and the pressure at the reactor inlet is frequently from 1 to 2 bar when using low steam dilution (corresponding to the BASF-Linde process) or from 3 to 8 bar when using high steam dilution (corresponding to the “steam active reforming process” (STAR process) of Phillips Petroleum Co., cf. U.S. Pat. No. 4,902,849, U.S. Pat. No. 4,996,387 and U.S. Pat. No. 5,389,342). Typical space velocities of propane over the catalyst (GHSV) are from 500 to 2 000 h-1, based on alkane to be reacted.

Dilution of the catalyst bed with catalytically inactive inert material leads to an increase in volume of the diluted catalyst bed compared to an undiluted catalyst bed. The larger reactor volume required as a result is preferably provided by lengthening the individual reactor tubes. An increase in the diameter of the reactor tubes is less preferred, since this reduces the surface area:volume ratio of the reactor, which acts against good heat transport. Increasing the number of reactor tubes while keeping the individual tubes at the same length is likewise less preferred, since this requires additional welds and connections which are costly. Lengthening the reactor tubes at a constant tube diameter results only in increased material costs and is therefore preferred. If desired, the abovementioned measures for increasing the reactor volume can be combined in order to achieve an optimum from both engineering and economic points of view.

The heat transmission coefficient of the reactor tubes is preferably >4 W/m² K, particularly preferably >10 W/m² K, in particular >20 W/m² K. Examples of suitable materials having such a heat transmission coefficient are steel and stainless steel.

The dehydrogenation-active catalyst is, for example, diluted with catalytically inactive inert material in the sections of the reactor in which the space-time yield without dilution if >7.0 kg/(kg_{bed}×h), based on alkene formed. As a result of the dilution, the space-time yield can be restricted to the abovementioned value as upper limit. This upper limit is preferably 4.0 kg/(kg_{bed}×h), particularly preferably 2.5 kg/(kg_{bed}×h) and especially 1.5 kg/(kg_{bed}×h). Due to the resulting lower incremental conversions, the establishment of high radial and/or axial thermal gradients is avoided. The catalyst can be diluted in the sections of the reactor in which the conversion without dilution would be >0.3 kg/(kg_{bed}×h), and it is preferably diluted in the sections in which the conversion without dilution would be >0.5 kg/(kg_{bed}×h), particularly preferably >1.0 kg/(kg_{bed}×h) and especially >1.5 kg/(kg_{bed}×h).

The dehydrogenation-active catalyst can also be applied as a shell to a shaped body made of catalytically inactive diluent material. Such shaped bodies may be rings or hollow extrudates which produce a low pressure drop in the catalyst bed.

In one embodiment of the process of the present invention, the catalyst bed is diluted with catalytically inactive inert material in sections of the reactor in which an internal temperature of >650° C., preferably >700° C. and particularly preferably >750° C., would occur in an undiluted catalyst bed of dehydrogenation-active catalyst during regeneration of the catalyst by burning-off of carbon deposits in an oxygen-containing gas.

Part of the heat required for the dehydrogenation can be generated in the catalyst bed itself by combustion of hydrogen, hydrocarbons and carbon with mixed-in oxygen. The combustion occurs catalytically. The dehydrogenation catalyst used generally also catalyzed the combustion of hydrocarbons and of hydrogen with oxygen, so that in principle no specific oxidation catalyst different from this is required. In one embodiment, the combustion is carried out in the presence of one or more oxidation catalysts which selectively catalyze the combustion of hydrogen with oxygen in the presence of hydrocarbons. The combustion of the hydrocarbons with oxygen to form CO and CO₂ then proceeds only to a minor extent, which has a favorable effect on the achieved selectivities to the formation of alkenes. The dehydrogenation catalyst and the oxidation catalyst are preferably present in different reaction zones.

The catalyst which selectively catalyzes the oxidation of hydrogen in the presence of hydrocarbons is preferably located at places at which the oxygen partial pressure is higher than at other points in the reactor, in particular in the vicinity of the point at which the oxygen-containing gas is fed in. The oxygen-containing gas and/or hydrogen can be introduced at one or more points in the reactor.

A preferred catalyst which selectively catalyzes the combustion of hydrogen comprises oxides or phosphates selected from the group consisting of the oxides and phosphates of germanium, tin, lead, arsenic, antimony and bismuth. A further preferred catalyst which catalyzes the combustion of hydrogen comprises a noble metal of transition group VIII or I.

The dehydrogenation catalysts used generally comprise a support and an active composition. The support is a heat-resistant oxide or mixed oxide. The dehydrogenation catalysts preferably comprise a metal oxide selected from the group consisting of zirconium dioxide, zinc oxide, aluminum oxide, silicon dioxide, titanium dioxide, magnesium oxide, lanthanum oxide, cerium oxide and mixtures thereof as support. Preferred supports are zirconium dioxide and/or silicon dioxide; particular preference is given to mixtures of zirconium dioxide and silicon dioxide.

The active composition of the dehydrogenation catalysts generally comprises one or more elements of transition group VIII, preferably platinum and/or palladium, particularly preferably platinum. In addition, the dehydrogenation catalysts may further comprise one or more elements of main groups I and/or II, preferably potassium and/or cesium. Furthermore, the dehydrogenation catalysts may comprise one or more elements of transition group m including the lanthanides and actinides, preferably lanthanum and/or cerium. Finally, the dehydrogenation catalysts may comprise one or more elements of main groups III and/or IV, preferably one or more elements from the group consisting of boron, gallium, silicon, germanium, tin and led, particularly preferably tin.

In a preferred embodiment, the dehydrogenation catalyst comprises at least one element of transition group VIII, at least one element of main groups I and/or II, at least one element of main groups III and/or IV and at least one element of transition group III including the lanthanides and actinides.

The alkane dehydrogenation is usually carried out in the presence of steam. The added steam serves as heat carrier and aids the gasification of organic deposits on the catalysts, thus countering carbonization of the catalysts and increasing the operating life of the catalyst. The organic deposits are converted into carbon monoxide and carbon dioxide.

The dehydrogenation catalyst can be regenerated in a manner known per se. Thus, steam can be added to the reaction gas mixture or an oxygen-containing gas at elevated temperature can be passed over the catalyst bed from time to time and the carbon deposits can be burnt off in this way.

Suitable alkanes which can be used in the process of the present invention have from 2 to 14 carbon atoms, preferably from 2 to 6 carbon atoms. Examples are ethane, propane, n-butane, isobutane, pentane and hexane. Preference is given to ethane, propane and butanes. Particular preference is given to propane and butane, and propane is especially preferred.

The alkane used in the alkane dehydrogenation does not have to be chemically pure. For example, the propane used can further comprise up to 50% by volume of additional gases such as ethane, methane, ethylene, butanes, butenes, propine, acetylene, H₂S, SO₂ and pentanes. The butane used can be a mixture of n-butane and isobutane and can further comprise, for example, up to 50% by volume of methane, ethane, ethene, propane, propene, propine, acetylene, C₅- and C₆-hydrocarbons and also H₂S and SO₂. The crude propane/crude butane used generally contains at least 60% by volume, preferably at least 70% by volume, particularly preferably at least 80% by volume, in particular at least 90% by volume and very particularly preferably at least 95% by volume, of propane or butane.

The alkane dehydrogenation gives a gas mixture comprising not only alkene and unreacted alkane but also secondary constituents. Usual secondary constituents are hydrogen, water, nitrogen, CO, CO₂ and cracking products of the alkane used. The composition of the gas mixture leaving the dehydrogenation stage can vary greatly. Thus, when the dehydrogenation is carried out with the introduction of oxygen and additional hydrogen, the product gas mixture will have a comparatively high content of water and carbon oxides. When no introduction of oxygen is employed, the product gas mixture from the dehydrogenation will have a comparatively high hydrogen content. For example, the product gas mixture leaving the dehydrogenation reactor in the dehydrogenation of propane comprises at least the constituents propane, propene and molecular hydrogen. However, it will generally further comprise N₂, H₂O, methane, ethane, ethylene, CO and CO₂. It will usually be under a pressure of from 0.3 to 10 bar and frequently have a temperature of from 400 to 700° C., in favorable cases from 450 to 600° C.

The invention is illustrated by the following examples.

### EXAMPLE 1

Production of the Catalyst

5 000 g of a crushed ZrO₂/SiO₂ mixed oxide from Norton (screen fraction: 1.6-2 mm) were impregnated with a solution of 59.96 g of SnCl₂.2H₂O and 39.43 g of H₂PtCl₆.6H₂O in 2 000 ml of ethanol corresponding to the solvent uptake. The composition was mixed in a rotating vessel at room temperature for 2 hours, subsequently dried at 100° C. for 15 hours and calcined at 560° C. for 3 hours.

The catalyst was then impregnated with a solution of 38.55 g of CsNO₃, 67.97 g of KNO₃ and 491.65 g of La(NO₃) which had been made up with water to a total volume of 2 000 ml corresponding to the water uptake. The catalyst was mixed in a rotating vessel at room temperature for 2 hours, subsequently dried at 100° C. for 15 hours and calcined at 560° C. for 3 hours.

The catalyst had a BET surface area of 84 m²/g.

### EXAMPLE 2

Dehydrogenation of Propane to Propene

125 ml, corresponding to 140.57 g, of the catalyst produced in example 1 were intimately mixed with 1 375 ml of steatite spears (diameter: 1.5-2.5 mm) and installed in a tube reactor having an internal diameter of 40 mm and a length of 180 cm. The 114.5 cm long catalyst bed was arranged so that the catalyst was located in the isothermal region of the electrically heated reactor tube. The remaining volume of the reactor tube was filled with steatite spheres (diameter: 4-5 mm). The reactor was heated to 500° C. (reactor wall temperature) at a nitrogen flow of 250 standard 1/h and a reactor outlet pressure of 1.5 bar.

The catalyst was supplied, in succession for 30 minutes in each case, at 500° C. firstly with diluted hydrogen (50 standard l/h of H₂+200 standard l/h of N₂), then with undiluted hydrogen (250 standard l/h of H₂), then with nitrogen for flushing (1 000 standard l/h of N₂), then with diluted air (50 standard l/h of air+200 standard l/h of N₂), then with undiluted air (250 standard l/h of air), then with nitrogen for flushing (1 000 standard l/h of N₂), then with diluted hydrogen (50 standard l/h of H₂+200 standard l/h of N₂) and subsequently with undiluted hydrogen (250 standard l/h of H₂).

250 standard l/h of propane (99.5% pure) and 250 g/h of water vapor were subsequently passed over the catalyst at 612° C. (reactor wall temperature). The reactor outlet pressure was 1.5 bar. The reaction products were analyzed by gas chromatography. After a reaction time of two hours, 47% of the propane used was converted into propene with a selectivity of 97%. After a reaction time of 10 hours, the conversion was 42% and the selectivity was 97%.

### COMPARATIVE EXAMPLE

125 ml, corresponding to 140.57 g, of the catalyst produced in example 1 were installed in a tube reactor having an internal diameter of 40 mm and a length of 180 cm. The 9.5 cm long catalyst bed was arranged so that the catalyst was located in the isothermal region of the electrically heated reactor tube. The remaining volume of the reactor tube was filled with steatite spheres (diameter: 4-5 mm). The reactor was heated to 500° C. (reactor wall temperature) at a nitrogen flow of 250 standard l/h and a reactor outlet pressure of 1.5 bar.

The catalyst was activated by means of hydrogen and air as described in example 2.

250 standard l/h of propane (99.5% pure) and 250 g/h of water vapor were subsequently passed over the catalyst at 612° C. (reactor wall temperature). The reactor outlet pressure was 1.5 bar. The reaction products were analyzed by gas chromatography. After a reaction time of two hours, 25% of the propane used was converted into propene with a selectivity of 96%. After a reaction time of 10 hours, the conversion was 24% and the selectivity was 97%.

1. An isothermal process for the dehydrogenation of alkanes to the corresponding alkenes over a catalyst bed comprising a dehydrogenation-active catalyst wherein heat is introduced from the outside into the reacting gas mixture by heating the reactor externally, and wherein the catalyst bed comprises a catalytically inactive, inert diluent material.

2. A process as claimed in claim 1, wherein the catalytically inactive, inert diluent material is selected from the group consisting of the oxides of elements of main groups II, III and IV, transition groups III and IV and V and mixtures thereof and nitrides and carbides of elements of main groups III and IV.

3. A process as claimed in claim 1, wherein the catalytically inactive, inert diluent material is selected from the group consisting of magnesium oxide, aluminum oxide, silicon dioxide, steatite, titanium dioxide, zirconium dioxide, niobium oxide, thorium oxide, aluminum nitride, silicon carbide, magnesium silicate, aluminum silicate, clay, kaolin, pumice and mixtures thereof.

4. A process as claimed in claim 1, wherein the catalytically inactive, inert diluent material has a BET surface area of <10 m²/g.

5. A process as claimed in claim 1, wherein the catalytically inactive, inert diluent material has a coefficient of thermal conduction of >0.04 W/(m×K).

6. A process as claimed in claim 1, wherein the space-time yield based on alkene formed is limited to 7.0 kg/(kg_{bed}×h) by the presence of the catalytically inactive diluent material in the catalyst bed.

7. A process as claimed in claim 1, wherein the catalytically inactive, inert diluent material is present in the form of shaped bodies selected from the group consisting of pellets and extrudates having an average diameter of from 2 to 8 mm, an average height of from 2 to 16 mm, with the height being from 0.5 to 4 times the diameter, rings and hollow extrudates having an average external diameter and an average height of from 6 to 20 mm, with the height being from 0.5 to 4 times the diameter and the wall thickness being from 0.1 to 0.25 times the diameter, and spheres having an average diameter of from 1 to 5 mm.

8. A process as claimed in claim 1, wherein the proportion of empty space in the bed is at least 30%.

9. A process as claimed in claim 1, wherein the dehydrogenation-active catalyst comprises one or more elements of transition group VIII, one or more elements of main groups I and/or II, one or more elements of transition group III including the lanthanides and actinides and one or more elements of main groups III and/or IV on an oxidic support.

10. A process as claimed in claim 1 carried out in a tube reactor or a shell-and-tube reactor.

11. A process as claimed in claim 1 in which propane is dehydrogenated.
